(19) United States
(12) Patent Application Publication
(10) Pub. No.: US 2017/0058319 A1
(43) Pub. Date: Mar. 2, 2017

(54) NANOSENSOR FOR ASSESSING THROMBIN INHIBITORS

(71) Applicant: VIRGINIA COMMONWEALTH UNIVERSITY, Richmond, VA (US)

(72) Inventors: Umesh R. Desai, Glen Allen, VA (US); Bruce D. Spiess, Manakin-Sabot, VA (US); Donald F. Brophy, Glen Allen, VA (US); Erika J. Martin, Richmond, VA (US); Kuberan Balagurunathan, Richmond, VA (US)

(21) Appl. No.: 15/123,079

(22) PCT Filed: Mar. 25, 2015
(86) PCT No.: PCT/US2015/022420

§ 371 Date: Sep. 1, 2016

Related U.S. Application Data
(60) Provisional application No. 62/036,857, filed on Aug. 13, 2014.
(60) Provisional application No. 61/970,628, filed on Mar. 26, 2014.

(51) Int. Cl. C12Q 1/56; G01N 33/86; G01N 21/64; G01N 33/542; C12N 9/74; G01N 33/58

(52) U.S. Cl. CPC C12Q 1/56; C12N 9/6429; C12Y 304/21005; G01N 33/582; G01N 21/6428; G01N 33/542; G01N 33/587; G01N 33/86; G01N 2333/974; G01N 2021/6439; G01N 2021/6432

(57) ABSTRACT

Constructs and monitoring systems to assess the level of molecules that bind to thrombin (e.g. thrombin regulators and inhibitors) are provided. The constructs are nanosensors comprising i) a thrombin molecule to which is bound a reporter ligand comprising a fluorescent label, ii) a fluorescence-quenching metal nanoparticle, and, optionally iii) a fluorescence-quenching dye molecule attached to one or both of the nanoparticle and the thrombin molecule. The binding of a thrombin regulator or inhibitor to the thrombin molecule displaces the reporter ligand, and the signal from the fluorescent label increases. The increase is proportional to the concentration of thrombin-binding molecule in the sample.

### CROSS-REFERENCE TO RELATED APPLICATIONS

This application claims benefit of U.S. provisional patent application 61/970,628, filed Mar. 26, 2014, and U.S. provisional patent application 62/036,857, filed Aug. 13, 2014, the complete contents of each of which are hereby incorporated by reference.

### STATEMENT OF FEDERALLY SPONSORED RESEARCH AND DEVELOPMENT

This invention was made with government support under grant # HL107152 awarded by the National Heart, Lung, and Blood Institute (NHLBI), a division of the National Institutes of Health. The United States government has certain rights in the invention.

### SEQUENCE LISTING

This application includes as the Sequence Listing the complete contents of the accompanying text file “Sequence.txt”, created Mar. 23, 2015, containing 4,096 bytes, hereby incorporated by reference.

### DESCRIPTION

### Background of the Invention

Field of the Invention

The invention generally relates to monitoring systems to assess in vitro, ex vivo and in vivo levels of proteins and/or drugs that interact with thrombin. In particular, the invention provides nanosensors that detect the presence of molecules that bind to thrombin, such as thrombin regulators and inhibitors.

Background of the Invention

Thrombin (also called factor IIa (fIIa)) is a complex serine protease involved in coagulation, inflammation, angiogenesis and cancer.¹⁻⁵ With regard to coagulation, thrombin is an amplification factor. In addition, it functions as a cell signaling protein for a wide range of cellular functions. Pro-thrombin (factor II (fII)) is produced by the liver and circulated in the serum in an inactivated form. When pro-thrombin is cleaved by activated factor Xa (fXa) it generates thrombin, which in turn binds to fibrinogen and forms soluble fibrin monomers that organize and generate an insoluble fibrin mesh. Cross-linking of the fibrin strands in the mesh generates clot. Therefore, thrombin is arguably the most important factor in the formation of a clot, which stops bleeding and begins the process of wound healing (see FIG. 1).⁵⁻⁹

Thrombin is also involved in a number of other reactions. It binds to thrombomodulin and the complex activates protein C, which in turn down regulates precursor proteins factors V and VIII of the coagulation cascade.^{5,10,11} Thus, thrombin also regulates its own formation and through this feedback process plays a key role in homeostasis. Another process that involves thrombin's catalytic activity is platelet activation. Thrombin is known to cleave platelet surface receptors called PARs, which are involved in the process of platelet activation, a fundamental process that is initiated in hemostasis and thrombosis.^{1,12-15} Thrombin is also recognized by platelet surface glycoprotein GPIb-alpha and the PAR-1 receptor, which also contribute to platelet activation.^{5,15} Platelets have the ability to further release thrombin in a neo-tissue and the “thrombin burst” is thought to be critical in the formation of a stable clot.^{16,17} Thrombin also activates the release of tissue plasminogen activator (TPA) from endothelial cells and platelets.⁵ TPA is the primary factor involved in dissolving fibrin strands in a process known as fibrinolysis. Thrombin also plays an important role in inflammation. In fact, coagulation is thought be an inflammation-related process.^{5,18} The associated reactions include cell signaling that upregulates the intracellular messenger NκKB¹⁸ via the release of ICAM-1, a protein that slows white cell and platelet rolling along endothelial surfaces. Therefore, thrombin causes the adherence of white cells to areas of the perturbed endothelium. Finally, thrombin has been proposed as an important factor that contributes to angiogenesis (the growth of new blood vessels), and regulating thrombin is known to effectively shut down tissue growth, which is especially important for cancer treatment.^{3,4}

As evident from the above discussion, regulators of thrombin are likely to be useful in a number of physiological and pathological processes. These include, but are not limited to, coagulation, fibrinolysis, inflammation, angiogenesis, and cancer. In fact, several inhibitors of thrombin are already used in the clinic to prevent excessive clot formation. These include antithrombin (AT), argatroban, bivalirudin, dabigatran, desirudin, heparins, hirudin, lepirudin, and others.^{5,19-21} These inhibitors either attack the active site, or an allosteric site of thrombin, or may also simultaneously attack both sites. Inhibitors that directly attack the active site include antithrombin, argatroban, and dabigatran. The heparins, which include unfractionated heparin (UFH), low molecular weight heparin (LMWH), ultralow molecular weight heparins (ULMWH), and heparin pentasaccharides (H5s) (fondaparinux (FPX), idraparinux (IPX) and others), utilize antithrombin to inhibit thrombin.^{21,22} This implies that the heparins indirectly target the active site through antithrombin. However, longer heparins also bind to thrombin's exosite 2, a highly charged allosteric site on thrombin surface, to help inhibit the enzyme. In contrast, the hirudins including hirudin, bivalirudin, lepirudin, desirudin and others, bind to exosite 1 of thrombin, another charged allosteric site, and induce inhibition.¹⁹ These agents also engage thrombin's active site and may be thought of as dual action inhibitors (competitive and non-competitive).²³

Antithrombin:

A classic method of regulating thrombin activity is through antithrombin (previously known as AT-III). AT is a circulating glycoprotein secreted by the liver with plieotropic effects. It is most known for its interaction with UFH, LMWH, ULMWH, and H5s that activate a key-in-lock mechanism inducing AT into a morphologic change. AT binding to UFH dramatically increases the reaction with a number of coagulation serine proteases resulting in 500-1,000-fold increase in the rate of reaction, especially for thrombin.²⁴ AT is secreted by the liver in a steady production phase dependent upon genetic predetermined programming, general health of the organism, liver blood flow, liver pathology, and infections and drug effects. Essentially AT production cannot be increased in response to need or loss of circulating levels/activity. Interestingly, despite this interconnectivity and its importance to physiology and pathology, there is no positive feedback loop that stimulates increased AT production. Likewise, neonates and young children, who do not have a mature liver production of AT, exhibit significant drops in AT circulating levels under unusual conditions such as an externally administered drug or high production of thrombin.²⁵

The plasma levels of AT are therefore crucial for optimal operation of a number of processes and AT can be thought of as a circulating buffer, especially for coagulation and inflammation. Plasma AT binds to the glycocalyxx of the vascular inner lining, which is a protective coating generated by endothelial cells and consists of mucous polysaccharides called glycosaminoglycans (GAGs) and include heparan sulfate, chondroitin sulfate and others. These GAGs present in the glycocalyxx coating bind to circulating AT and activate it, as described above.^{21,22,24} In the natural healthy state, the endothelium thereby produces a consistent anti-thrombotic and anti-inflammatory barrier.^{25,27} The activation of AT by heparan is only one of a number of normal and anti-thrombotic mechanisms exercised by the endothelium. Others include the active secretion of nitric oxide as well as tissue plasminogen activator, thrombomodulin, protein C and protein S.

Heparins:

Heparin is not normally found circulating free in the plasma (nor is heparan sulfate). Heparin differs from heparan sulfate in terms of the chain length of the polysaccharides as well as the level of sulfate groups and substitution pattern.^{21,22,28} Heparin is synthesized in the granules of mast cells (contained in specific tissues) and is released upon stimulation of an external assault. Mast cells are present in high concentrations within the tissues of the lung and intestine, which are areas exposed to the outside world. Heparin released from mast cells is an inflammatory mediator which increases white cell movement through tissues. Heparin and other GAGs have numerous functions including roles in angiogenesis, inflammation promotion and wide range of cell signaling.^{29,30}

Unfractionated heparin (UFH): Cardiac surgery since the late 1950's has depended upon the intravascular injection of UFH isolated from swine gut or bovine lung into the veins of humans. As one might expect, since UFH is unnatural to human plasma a number of adverse responses occur. Perhaps most importantly, but greatly understudied is the fact that UFH leads to release of heparan sulfate from native endothelium.^{25,31} UFH injection leads the native endothelium open for attack by inflammatory white cells, platelets and serine proteases leading to increased risk of vasospasm, inflammation and coagulation/thrombosis. Indeed if one wishes to release heparan sulfate from endothelium in the laboratory setting one exposes it to UFH.

UFH is given in large dosages prior to cardiopulmonary bypass (CPB). The result is a near total conversion of AT to activated AT, which is available for immediate inhibition of any activated serine proteases, complement factor or bradykinin.²⁵ The infusion of UFH also leads to the native circulating platelets becoming activated. UFH stimulates the externalization of PF-4 binding ligands.^{25,32,33} These protein binding sites are normally held internally on the platelet cell membrane. Once a platelet detects free heparan sulfate or UFH it expresses these binding sites, as the PF-4 ligand is the natural method for the body to scavenge free anti-coagulants. This is a homeostatic mechanism intended to keep small vascular injuries from spreading beyond a localized region of insult.^{25,32,33} When UFH is administered as a pharmaceutical, the body's platelets are immediately placed on high alert and this in turn creates cell signaling to white cells, cytokine production and antibody production. The PF-4 heparin complex is highly antigenic and in 30-40% of heart patients, antibodies form to PF-4 heparin. By day 5-7 after CPB, these antibodies can have a deleterious effect. If present before heart surgery, they can lead to deadly thrombotic reactions known as heparin-induced thrombotic thrombocytopenic purpura (HITT).^{25,32-36} UFH is the most commonly received medication for all hospitalized patients and HITT is typically under identified, although it can cause morbidity and mortality. A characteristic salutary feature of HITT is low AT levels.³⁷

Direct Thrombin Inhibitors (DTIs):

Although heparin is the most widely prescribed intravenous drug in the United States today other anti-coagulants are rapidly coming on the market and finding niche usages. Peptide and peptidomimetic agents that are direct thrombin inhibitors are being developed and marketed for both IV and oral use. These agents include the intravenous agents hirudin, argatroban and bivalirudin; and the oral agents ximelagatran and dabigatran. DTIs prevent thrombin from interacting effectively with its normal substrates, resulting in reduced fibrin formation. DTIs also prevent thrombin-mediated feedback activation of factors V, VIII, III and XI. This multi-targeting effect has led to the assumption that DTIs would be highly effective in preventing a hypercoagulable state. DTIs including hirudin, bivalirudin, argatroban, melagatran (ximelagatran's active form) and dabigatran specifically target either the active site and/or exosite I of thrombin and function as competitive and/or mixed inhibitors. The peptides use an Arg at the P-1 position to target Asp¹⁸⁹. In addition, the P-2 and P-3 positions in the thrombin recognition sequence are usually hydrophobic amino acids such as Pro and Phe, respectively. The peptidomimetics typically contain a guanidine (or an amidine) moiety to mimic the P-1 Arg side chain and an organic scaffold such as an alicyclic, aromatic or heteroaromatic ring connected through amide or sulfonamide linker(s) to mimic the Pro and Phe residues at P-2 and P-3 positions. DTIs that do not have a guanidine or an amidine group use a basic amine, e.g., an alkyl amine, as a thrombin anchor. Exclusive exosite I ligands appear to induce some catalytic dysfunction of the enzyme. In addition, they compete with natural substrates, e.g., fibrinogen, to induce an anticoagulant effect.

Dabigatran etexilate is the most recent oral anticoagulant to reach the clinic for the treatment and prevention of venous thromboembolism in elective hip or knee surgeries. With respect to safety of this new drug, doses as high as 300 mg twice a day for 12 weeks have been evaluated with no abnormal liver function tests. However, in a trial with and without aspirin, approximately 11% of patients developed significant bleeding.²¹

DTIs are the drugs of choice for treating heparin induced thrombocytopenia/thrombosis (HIT/HITT) and they are frequently administered intravenously in intensive care units to prevent/treat thrombosis. In addition, bivalirudin is currently the drug of choice for use during cardiac stent placement, and the use of oral DTIs has been added to that of other anti-platelet agents for the outpatient treatment of patients with stents and their use for anti-coagulation pertaining to atrial fibrillation is also on the rise.

Unfortunately, each of these thrombin inhibitors has the potential to cause deleterious side effects, some of which can be lethal. One way to assess and greatly reduce such adverse events would be to monitor the level of inhibitor in a patient's circulation, preferably on a real-time basis using a readily obtainable sample, e.g. plasma. Unfortunately, up to now, no such assay exists for most inhibitors.

Assays are available for detecting AT. However, current technologies utilize ELISA or liquid chromatography. These techniques are moderate to complex in nature, are expensive to use, and trained technicians are required to run and maintain the instrumentation that is involved. The individual tests take about 30 minutes to run, but in the context of present human medical care systems, at best, AT tests results are obtained in approximately a one hour time frame. Further, many hospitals do not have AT tests available 24 hours a day or on a STAT basis. Unfortunately, these factors have limited the use of AT testing, and it is not routinely performed for patients who are receiving intravenous heparin therapy, even those having heart surgery. This is extremely unfortunate; it is well-established that that the incidence of acquired AT deficiency due to heparin infusion in hospital patients can be as high as 25%. During heart surgery, heparin infusion routinely reduces the levels of AT to levels seen in sepsis and shock and the incidence of liver failure due to low levels of AT is approximately 5%. AT replacement therapy might prevent these untoward complications. However, physiological levels of AT must be maintained within a very narrow range and administration must be carefully controlled and monitored. Past experimental drug protocols using AT have primarily utilized dosage based only upon patient weight, which easily leads to overdosing or underdosing a patient. Clearly, the limitations of the present day technology discussed above have hampered the use of AT testing in patients receiving heparin therapy and also in patients who might otherwise benefit from AT replacement therapy.

There is clearly a need in the art for rapid, sensitive and cost effective technology to detect and measure levels of molecules that bind to thrombin, e.g. thrombin inhibitors such as AT.

Kwon et al. (U.S. Pat. No. 8,551,727 B2) disclose a nanoprobe designed to detect the presence of a protease such as thrombin. The nanoprobe comprises a fluorophore covalently linked to a peptide, the sequence of which is specific for and cleaved by the protease that is targeted for detection. However, the detection of thrombin inhibitors is not described, and the use of thrombin and thrombin-based peptides to detect thrombin inhibitors, is also not described.

In summary, thrombin is a critical component of the coagulation cascade, and arguably the most important factor. It is required for induction of the cascade that culminates in fibrin clot formation, and also controls a regulatory feedback to down-regulate clotting and remove fibrin after a clot is stabilized and during the healing process. Due to the critical nature of the clotting process, there is redundancy in protein factors that are involved in the processes of clotting, which also includes “unclotting”. Diseases and pharmaceutical treatments can interfere with either process, and frequently do so by inhibiting thrombin. There is a need in the art to rapidly and reliably detect the presence of thrombin inhibitors e.g. in biological samples from patients being treated with such inhibitors.

### SUMMARY OF THE INVENTION

The present invention provides constructs and methods of using the constructs to detect molecules that bind thrombin, e.g. thrombin regulators, inhibitors, drugs, etc. in samples. The constructs comprise a “thrombin molecule” comprising at least one binding site for a molecule that binds thrombin, e.g. a thrombin inhibitor. The “thrombin molecule” may be thrombin per se or a molecule that is based on or derived from (a “variant” of) thrombin, as described elsewhere herein. The thrombin molecule is attached to a nanoparticle, either directly (e.g. via a chemical linkage such as a disulfide or amide bond) or via a linker (a “tether” or “spacer”) which joins the thrombin molecule to the nanoparticle. Certain nanoparticles are capable of quenching fluorescence. A reporter ligand comprising a fluorescent label is reversibly bound to the thrombin molecule at one or more binding sites. In the construct, the signal from the fluorescent label is attenuated or quenched due to its close proximity to one or more quenchers, which may be the nanosurface or a molecule conjugated to the nanosurface or a molecule covalently attached to thrombin. Thus, the construct, when intact, produces a fluorescent signal, which may or may not be detected, due to the attached quencher(s). When the construct comes into contact with a molecule which binds thrombin, e.g. an inhibitor of thrombin, which binds at the site where the reporter ligand is bound or to a distal binding site that is allosterically connected the reporter ligand binding site, the thrombin inhibitor displaces the non-covalently bound reporter ligand from its thrombin bound state, either by direct competition, or by initiating an allosteric change in at least one binding site. The reporter ligand is released from the thrombin molecule and the fluorescent signal from the fluorescent label is no longer quenched. As a result, the level of fluorescence emanating from the construct changes, or preferably increases. In a sample comprising a plurality of constructs, the collective increase in fluorescence that occurs when a plurality of thrombin-binding molecules, alternatively inhibitors or regulators, are present in the sample is detectable (measurable) and is positively correlated with the amount (level, number, etc.) of the thrombin-binding molecules, alternatively inhibitors or regulators. In other words, the fluorescence change is an indirect surrogate marker for the level of thrombin-binding molecule in the sample.

In addition, a second type of fluorescence-quenching molecule may be present in the constructs in that one or more quenching dye molecules may be attached. A dye molecule may be attached to the nanoparticle, or to the thrombin molecule, or a dye molecule may be attached to the nanoparticle and a second dye molecule may be attached to the thrombin molecule. The presence of the additional quencher(s) provides further contrast between the fluorescence produced by an intact construct, and the fluorescence produced by a construct from which the reporter ligand has dissociated.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

It is an object of this invention to provide nanoprobes comprising: a metal nanoparticle; a thrombin molecule attached to said metal nanoparticle, said thrombin molecule comprising at least one ligand binding site; and a reporter ligand non-covalently bound to said at least one ligand binding site, said reporter ligand comprising a fluorescent label; wherein an absorbance spectrum of said metal nanoparticle overlaps an emission spectrum of said fluorescent label; and wherein a distance between said metal nanoparticle and said fluorescent label allows quenching of a fluorescence signal from said fluorescent label by said metal nanoparticle. In some aspects, the thrombin molecule is covalently attached to said metal nanoparticle via a first linker molecule. In other aspects, the nanoprobes comprise a dye molecule attached to said metal nanoparticle, or a dye molecule attached to said thrombin molecule, or a dye molecule attached to said metal nanoparticle and a dye molecule attached to said metal nanoparticle, wherein the attachment is direct (e.g. via a covalent bond) or via a linker molecule, and wherein an absorbance spectrum of said dye molecule overlaps an emission spectrum of said fluorescent label, and wherein a distance between said dye molecule and said fluorescent label allows quenching of said fluorescence signal from said fluorescent label by said dye molecule. In some aspects, the reporter ligand comprises hirudin or a thrombin-binding portion of hirudin. In additional aspects, the fluorescent label is selected from the group consisting of fluorescein, cyanine, tetramethylrhodamine, and boron-dipyrromethene (BODIPY®). In yet other aspects, the metal nanoparticle is selected from the group consisting of gold, iron, copper, silver, platinum, tungsten and alloys and derivatives thereof. The thrombin molecule may comprise at least a portion of a thrombin protein sequence from a species selected from the group consisting of human, bovine, ovine, porcine, non-human primate and rodent. In addition, the thrombin molecule may comprise at least a portion of a thrombin protein sequence selected from the group consisting of an alpha thrombin, a beta thrombin and a gamma thrombin. In some aspects, the at least one binding site is selected from the group consisting of a thrombin catalytic active site, thrombin exosite 1, and thrombin exosite 2. In one aspect, the metal nanoparticle is gold, said thrombin molecule is bovine alpha thrombin, and said fluorescent label is fluorescein.

The invention also provides methods of detecting a thrombin inhibitor in a biological sample, comprising the steps of i) contacting said biological sample with a nanoprobe, said nanoprobe comprising: a metal nanoparticle; a thrombin molecule attached to said metal nanoparticle, said thrombin molecule comprising at least one ligand binding site; and a reporter ligand non-covalently bound to said at least one ligand binding site, said reporter ligand comprising a fluorescent label; wherein an absorbance spectrum of said metal nanoparticle overlaps an emission spectrum of said fluorescent label; and wherein a distance between said metal nanoparticle and said fluorescent label allows quenching of a fluorescence signal from said fluorescent label by said metal nanoparticle; and ii) detecting the presence or absence of a fluorescent signal from said fluorescent label, wherein in the presence of a fluorescent signal indicates that a thrombin inhibitor is present in the biological sample, and the absence of a fluorescent signal indicates that a thrombin inhibitor is not present in the biological sample. In some aspects, the thrombin inhibitor detected in said detecting step is selected from the group consisting of antithrombin (AT), alpha-1-antitrypsin (α-1A), antiplasmin, heparin cofactor II, heparin, hirudin, desirudin, argatroban, bivalirudin, ximelagatran, melagatran, dabigatran, heparin, heparansulfate, unfractionated heparin (UFH), low molecular weight heparin (LMWH), ultralow molecular weight heparins (ULMWH), heparin pentasaccharide (H5), fondaparinux (FPX), idraparinux (IPX) and a synthetic heparin analog. In other aspects, the detecting step further comprises a step of quantifying said thrombin inhibitor. The step of quantifying may include the steps of measuring said fluorescent signal and comparing measured fluorescence to one or more reference values. In some aspects, the metal nanoparticle is selected from the group consisting of gold, iron, copper, silver, platinum, tungsten and alloys and derivatives thereof. In additional aspects, the fluorescent peptide is selected from the group consisting of fluorescein, cyanine, tetramethylrhodamine, and boron-dipyrromethene (BODIPY®). In some aspects, the thrombin molecule is from a species selected from the group consisting of human, bovine, ovine, porcine, non-human primate and rodent. In further aspects, the thrombin molecule comprises at least a portion of a thrombin protein sequence selected from the group consisting of an alpha thrombin, a beta thrombin and a gamma thrombin. In additional aspects, the at least one ligand binding site is selected from the group consisting of a thrombin catalytic active site, thrombin exosite 1, and thrombin exosite 2. In some aspects, the metal nanoparticle is gold, said thrombin molecule is bovine alpha thrombin, and said fluorescent peptide is fluoroscein. The method may be performed in vivo, ex vivo or in vitro. In certain aspects of the method, the nanoprobe further comprises a dye molecule attached to said metal nanoparticle, or a dye molecule attached to said thrombin molecule, or a dye molecule attached to said metal nanoparticle and a dye molecule attached to said metal nanoparticle, wherein the attachment may be either direct (e.g. via a covalent bond) or indirect e.g. via a linker molecule; and wherein an absorbance spectrum of said dye molecule overlaps an emission spectrum of said fluorescent label, and wherein a distance between said dye molecule and said fluorescent label allows quenching of said fluorescence signal from said fluorescent label by said dye molecule.

### BRIEF DESCRIPTION OF THE DRAWINGS

FIG. 1. Some of the well-established functions of thrombin. Image taken from Lichtman's Atlas of Hematology, The McGraw-Hill Companies, Inc, Columbus, Ohio, 2007.

FIG. 2. Schematic illustration of an exemplary embodiment of the invention.

FIG. 3. Absorbance spectrum of Au—NHNH₂ nanoparticles (NPs) in water at room temperature.

FIG. 4. Absorbance spectrum of Au-thrombin nanoparticles (AU-TH NPs) in water at room temperature.

FIG. 5. Stability of Au-TH NPs at room temperature.

FIG. 6. Standard curve of velocity of substrate hydrolysis compared to different thrombin concentrations. Extrapolation of the velocity obtained for the Au-TH NP can be used to find the amount of thrombin loaded on the surface of the Au—NP.

FIG. 7. Michaelis-Menten kinetics analysis of free thrombin as compared to Au-TH NP shows that surface bound thrombin has catalytic efficiency identical to that of free thrombin.

FIG. 8. Reactivity of Au-TH NP towards antithrombin (AT) shows that AT can neutralize surface bound thrombin up to 60% alone and up to 70% in the presence of heparin.

FIG. 9. Quenching of HirP fluorescence upon binding to Au-TH nanoprobe.

FIG. 10. Antithrombin concentration based release of hirudin fluorescence.

FIG. 11. Release of hirudin fluorescence following incubation with human plasma. Corresponding increases were not observed following incubation with bovine serum albumin (BSA).

FIGS. 12A and B. Schematic representation of an exemplary aspect of the invention.

FIGS. 13A and B. Schematic representation of an exemplary aspect of the invention.

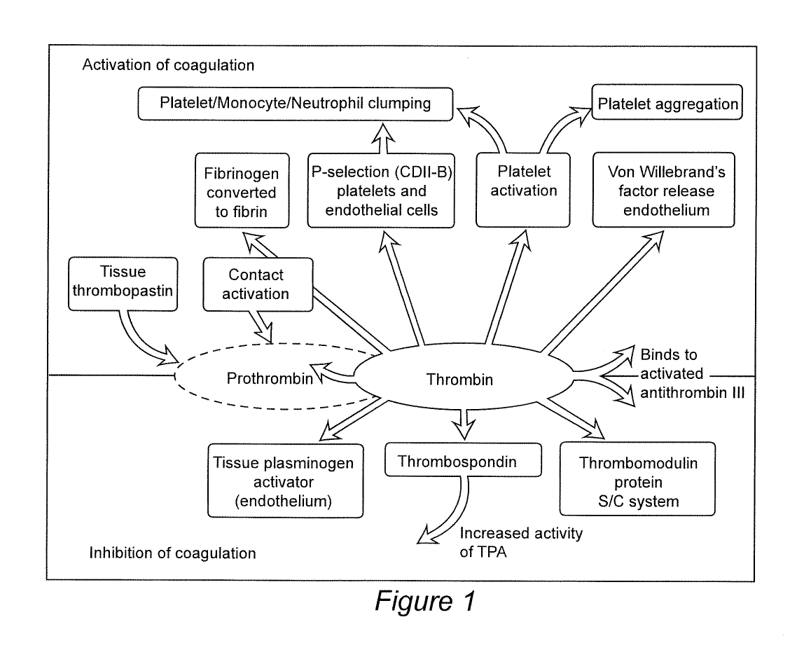

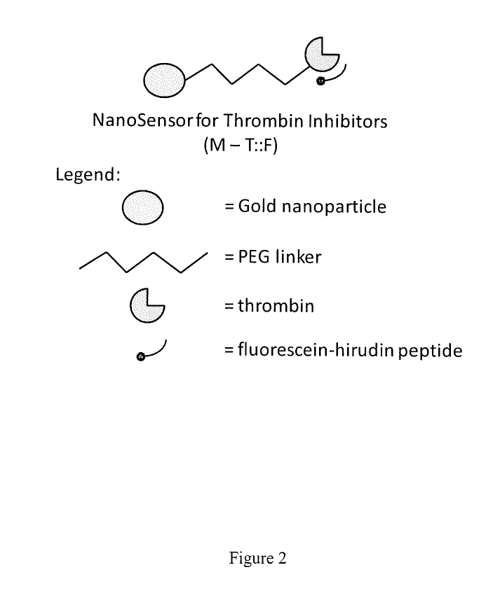

### DETAILED DESCRIPTION

Provided herein are constructs, compositions comprising the constructs and methods of using the constructs for detecting and quantitating molecules that bind to thrombin, e.g. proteins such as thrombin inhibitors, various thrombin regulators, synthetic drugs, etc., in a sample of interest. In one aspect, when fully operational, the constructs comprise: i) a thrombin molecule with an associated reporter ligand and ii) a nanoparticle, and, optionally, iii) a linking molecule that joins the thrombin molecule to the nanoparticle. In other aspects, when fully operational, a fluorescence-quenching dye molecule is associated with one or both of the thrombin molecule and the nanoparticle. The dye molecule is optionally attached via a linker.

### The Thrombin Molecule

By “thrombin molecule (TH)” we mean a full length alpha-thrombin molecule ((The refined 1.9 A crystal structure of human alpha-thrombin: interaction with D-Phe-Pro-Arg chloromethylketone and significance of the Tyr-Pro-Pro-Trp insertion segment. W Bode, I Mayr, U Baumann, R Huber, S R Stone, and J Hofsteenge; EMBO J. (1989) vol 8; issue 11; pgs 3467-3475; Human thrombins. Production, evaluation, and properties of alpha-thrombin. J. W. Fenton 2^{nd}; M. J. Fasco and A. B. Stackrow; J. Biol. Chem. (1977) vol 252; pgs 3587-3598)) or a portion of a full-length thrombin molecule that retains the proteolytic activity, as exemplified by beta-thrombin or gamma-thrombin ((Crystallographic structure of human gamma-thrombin. T J Rydel, M Yin, K P Padmanabhan, D T Blankenship, A D Cardin, P E Correa, J W Fenton, 2nd and A Tulinsky; J. Biol. Chem. (1994) vol 269; pgs 22000-22006)). The thrombin molecule may be isolated from a natural source, or may be a synthetic or recombinant protein or fragment. The TH has at least one ligand binding site and may bind to (have affinity for) one or more than one ligand. The binding may be selective or specific. The thrombin molecule may be of any type from any species, e.g. human, bovine, ovine, porcine, murine, lapine, equine, canine, or other similar species. Any type of TH or TH derivative may be used, so long as the TH molecule comprises at least one binding site for a ligand of interest, i.e. a ligand which is to be detected by the constructs of the invention, such as a thrombin inhibitor. In some aspects, the binding site is a naturally occurring binding site that is found in thrombin, e.g. the active site, exosite 1, exosite 2, or the sodium binding site. One example of alpha-thrombin (EC 3.4.21.5) is from human species and refers to the amino acid sequence

(SEQ ID NO: 1)
1-IVEGSDAEIGMSPWQVMLFRKSPQELLCGASLISDRWVLTAAHCLLYP
PWDKNFTENDLLVRIGICHSRTRYERNIEKISMLEKIYIHPRYNWRENLD
RDIALMKLKKPVAFSDYIHPVCLPDRETAASLLQAGYKGRVTGWGNLKET
WTANVGKGQPSVLQVVNLPIVERPVCKDSTRIRITDNMFCAGYKPDEGKR
GDACEGDSGGPFVMKSPFNNRWYQMGIVSWGEGCDRDGKYGFYTHVFRLK
KWIQKVIDQFGE-259,

which contains all four ligand binding sites including the active site, exosite 1, exosite 2 and the sodium binding site. For each of these, the TH can be alpha, beta or gamma thrombin.

In one aspect, the TH is of alpha type, the amino acid sequence of which is set forth in SEQ ID NO: 1. Other exemplary TH that may be used in the practice of the invention include but are not limited to: gamma thrombin, the amino acid sequence of which is set forth in SEQ ID NO: 2:

(1-GQPSVLQVVNLPIVERPVCKDSTRIRITDNMFCAGYKPDEGKRGDAC
EGDSGGPFVMKSPFNNRWYQMGIVSWGEGCDRDGKYGFYTHVERLKKWIQ
KVIDQFGE-105,SEQ ID NO: 2).

Those of skill in the art will recognize that the exact sequences listed above can be modified within the bounds of the invention so as to provide variants or derivatives of the thrombin sequences which are suitable for use in the constructs described herein. For example, conservative amino acid replacements and/or various substitutions and/or deletions and/or insertions of one or more residues, may be made, e.g. up to about 10 (1-10) amino acids can be deleted from or added to the carboxy and/or amino termini, so long as the ability or activity of the resulting variant to bind a reporter ligand and a thrombin-binding molecule is not impaired. Further, active polypeptide or peptide fragments or portions of the thrombin molecules disclosed herein may also be employed, as long as at least one ligand binding site is retained, and so long as a reporter ligand can bind to the at least one binding site and be displaced therefrom by the binding of a molecule of interest that binds thrombin, as described herein. Generally, such sequences have at least about 50 sequence identity to the sequences set forth herein, e.g. at least about 50, 60, 70, 80, 85, 90, 95 or greater (e.g. 96, 97, 98 or 99% identity) to the sequences described or set forth herein, including alpha, beta and gamma thrombin from human or other species.

In other aspects, the binding site to which the reporter ligand binds is not a “natural” thrombin binding site. Instead, the binding site may be genetically engineered into the thrombin molecule, e.g. a recombinant thrombin molecule which comprises one or more ligand binding sites not naturally found in thrombin may be used. Such binding sites are selected so as to have ligands that are suitable for use in the present invention, e.g. ligands that can be fluorescently labeled and can bind to and be displaced from the binding site, either by allosteric changes in binding site topography that occur when another molecule of interest binds elsewhere on the recombinant thrombin molecule, or directly via competitive displacement by another ligand of interest with a higher affinity for the binding site.

### The Reporter Ligand and its Binding to a Site on Thrombin

A reporter ligand binds non-covalently and reversibly (detachably, removably) to the at least one binding site of the thrombin molecule in a construct. The reporter ligand binds to TH specifically or with high specificity. Thus, when the constructs of the disclosure are utilized, specific thrombin-binding molecules can be distinguished from non-specific thrombin binding molecules, since the reporter ligands employed will not be displaced by molecules that bind thrombin non-specifically. At least one binding site may occur naturally in thrombin, e.g. the active site, exosite 1, exosite 2, Na binding site, etc., and the reporter ligand which binds to this naturally occurring binding site may be a ligand of thrombin (e.g. a thrombin inhibitor or other protein or polypeptide or peptide molecule that is known to bind thrombin either in vivo or in vitro), which may be a naturally occurring ligand or derived from or based on a naturally occurring ligand. Suitable reporter ligands include but are not limited to the hirudin polypeptide

(1-VVYTDCTESGQNLCLCEGSNVCGQGNKCILGSDGEKNQCVTGEGTPK
PQSHNDGDFEEIPEEY(OSO₃H)LQ, SEQ ID NO: 3)

and peptide fragments thereof that retain thrombin binding activity, such as a fragment that includes C-terminal amino acids 54 to 65

(GDFEEIPEEYLQ SEQ ID NO: 4)

or variants of this sequence that include modifications of specific amino acid residues such as sulfation (OSO₃H) of tyrosine (Y); various hirudin-based anticoagulant pharmaceutical products, such as lepirudin (Refludan), hirudin derived from Hansenula (Thrombexx, Extrauma) and desinidin (Revasc/Iprivask), and other direct thrombin inhibitors derived chemically from hirudin; or variegin, a peptide inhibitor obtained from the tropical bont tick, etc.

Another group of reporter ligands include heparin and variants based on heparin structure including unfractionated heparin, low molecular weight heparins, heparin oligosaccharides (disaccharides, tetrasaccharides, etc) which have been covalently modified with appropriate fluorophores reported in the literature including 2-amino acridone, dansyl, fluorescein, etc. and which bind reversibly to exosite 2 of thrombin molecule.

Active derivatives or variants of these may be used, so long as the derivatives/variants exhibit a suitable binding affinity for the reporter ligand binding site(s). Further, reporter ligands may also be wholly synthetic in nature, e.g. purposefully designed to bind to the at least one binding site based on the structures of hirudin or heparin-based reporter ligands discussed above. Such ligands are may also be referred to as “small molecule” inhibitors, or regulators or modulators or ligands.

In other aspects, the binding site to which the reporter ligand binds is not a “natural” thrombin binding site. Instead, the binding site is genetically engineered into a thrombin molecule. In other words, a recombinant thrombin molecule which comprises one or more ligand binding sites not naturally found in thrombin may be used. Such binding sites are selected so as to have ligands that are suitable for use in the present invention, e.g. ligands that can be labeled with a detectable label and can bind to and be displaced from the binding site, either by allosteric changes in binding site topography that occur when another molecule of interest binds elsewhere on the recombinant thrombin molecule, or directly via competitive displacement by another ligand of interest with a higher affinity for the binding site. In this aspect, allosteric displacement may be of particular interest in that the engineered binding site may be a modification of a binding site that occurs naturally in thrombin (e.g. to increase or to decrease the affinity of the reporter ligand), or may be a binding site that occurs naturally in another different molecule (or a modified form thereof) so that the “thrombin molecule” is a fusion or chimeric polypeptide. All such aspects are encompassed in the present invention, so long as the resulting construct functions as described herein.

Reporter ligands generally bind to the at least one binding site with an affinity that is in a range of from about 10 femtomolar to about 10 millimolar, and generally is in the range of from about 10 nanomolar to about 10 micromolar or from about 20 nanomolar to 100 nanomolar. The affinity may vary, depending on the type of displacement that is required to detect a particular thrombin-binding molecule of interest (e.g. allosteric or competitive) and the binding affinity of the thrombin-binding molecule of interest.

### Detectable Labels

In the constructs, a detectable label is associated with (attached to, bound to, covalently or non-covalently bonded to, etc.) the reporter ligand. In other words, the reporter ligand is labeled with a detectable label that is generally covalently attached to the reporter ligand. The detectable label is generally a fluorescent label (a fluorophore). The reporter ligand in association with a fluorophore may be referred to as a “fluoroprobe”. The fluorescent signal from the label is “quenchable” or susceptible to quenching, i.e. is lessened when the label is located in proximity to one or more suitable molecules whose absorbance spectra overlap the emission spectrum of the label, such as a suitable metal, a dye molecule, etc.

Many fluorescent labels are known in the art and may be employed to label the reporter ligand, including but not limited to: aminoacridone-based dyes (e.g., 2-aminoacridone), fluorescein-based dyes (5-carboxyfluorescein), cyanine dyes, rhodamine dyes (e.g., tetramethylrhodamine), oxazine dyes, BODIPY® dyes (e.g., boron-dipyrromethene (BODIPY®)), etc. In some aspects, fluorescein-coupled to a hirudin-derived sequence, e.g., 5-(carboxy)-fluorescein-hirudin-54-65 containing Tyr63-OSO₃H, is used as the fluoroprobe.

### Nanoparticles

The constructs described herein comprise a “nanoparticle”. Nanoparticles are generally understood to be particles of less than about 250 nanometers in size. In nanotechnology, a “particle” is defined as “a small object that behaves as a whole unit with respect to its transport and properties”. Herein, if a linker is present in a construct, “nanoparticle” may refer to the nanoparticle per se without including the thrombin molecule and the linker. Alternatively, “nanoparticle” may refer to the nanoparticle plus the thrombin molecule, e.g. if the thrombin molecule is linked or bonded (e.g. covalently bonded) directly to the nanoparticle and no linker is present between the two entities.

In the present constructs, the nanoparticle component has the ability to attenuate or lessen (quench) a detectable signal emitted from a signaling molecule when the signaling molecule is located at a suitable distance from the nanoparticle (e.g. from about 0 to about 500 angstroms, or from about 5 to about 100 angstroms, or from about 5 to about 50 angstroms). In some aspects, the nanoparticle is a metal nanoparticle that is capable of quenching a fluorescent signal (the absorbance spectrum of the metal overlaps at least a portion of the emission spectrum of the fluorophore). Examples of suitable metals that may be used to form the nanoparticle include but are not limited to: gold, iron, copper, silver, platinum, tungsten, palladium, cobalt, tin, molybdenum, etc. and alloys thereof. In some aspects, the nanoparticles are formed from a magnetic metal such as iron, as this facilitates particle and construct isolation and manipulation. The average size of the nanoparticles that are employed is in the range of from about 1 nm to about 200 nm, and is usually in the range of from about 10 nm to about 50 nm.

When designing and manufacturing a particular type of construct, it is necessary to select fluorophore-metal pairs whose emission and absorption spectra overlap. Suitable pairings include but are not limited to: gold-fluorescein, gold-BODIPY®, silver-fluorescein and silver-BODIPY®.

### Linker Molecules

As discussed above, in some aspects, the thrombin molecule is linked directly (e.g. covalently attached or bonded) to the nanoparticle so only one or a very few (e.g. 2-4) atoms are present between the two, and in effect, no linker is present. In other aspects, at least one longer linker (e.g. 5 or more atoms in length) is present in the constructs described herein. A “linker” or “spacer” or “tether” refers to atoms that are present between two entities such as a thrombin molecule and a nanoparticle, and/or between a nanoparticle and a dye particle. The linker is present between two entities so that both entities are covalently linked to the linker molecule but at opposite ends thereof. Such linkers often are in the form of a chain of atoms, the purpose of which is to connect two entities, holding them in proximity to each other, but also to separate them by a desired or appropriate distance (understanding that some flexibility is present in the linking chain). The linkers are generally non-reactive, i.e. they do not readily react with atoms or molecules present in the media that is used for the assay, or with biological molecules that are present in biological samples, and they do not react with the molecule that is being detected (a molecule that binds to thrombin). In other words, cleavage or degradation of the tether (e.g. by thrombin or another protease) is not required and in fact, should not occur for optimal operation of the nanosensor. If two linkers are present in a construct, they may be the same or different.

The linking or spacing molecule may be of any suitable type and may be a cross-linking molecule, reactive ends of which react and form covalent bonds with the thrombin molecule and the nanoparticle. In various exemplary aspects, the linker is: an alkyl chain —(CH₂)ₙ— where n ranges from about 2 to about 30; an ethylene glycol chain of the type —(OCH₂CH₂)ₙ—, where ranges from about 2 to about 30; and an alkyl chain —(CH₂)ₙ-triazole-(CH₂)ₘ—, where n and m range from about 2 to about 15, and triazole is a five-membered cyclic ring with three nitrogens; various peptide spacers e.g. glycine-serine spacers, and/or peptide spacers that include one or more D amino acids; peptidomimetic spacers; etc. Additional exemplary linking molecules include but are not limited to: heterobifunctional chemical linkers, homobifunctional chemical linkers, polyethylene glycol (PEG)-based linkers, and nucleic acid based linkers. In some embodiments the synthetic linker is PEG or a functionalized PEG, for example, OPSS-SPEG-SVA (MW:5000) (Laysan Bio, Arab, Ala.). In some embodiments, thiol-functionalized PEG can be used. Other options for linkers include siloxanes (i.e., “chemically functionalized silica”), polysiloxanes, polyvinyl alcohol (PVA), polyvinyl acetate (PVAc), the polyacrylates and polymethacrylates, fluoropolymers, dendrimers, dextrans, cellulosic materials, and the like. In one embodiment the linker is a bifunctional chemical linker that is heterobifunctional. Suitable heterobifunctional chemical linkers include without limitation sulfoSMCC (Sulfosuccinimidyl-4-(N-maleimidomethyl)cyclohexane-1-carboxylate), and 1c-SPDP (N-Succinimidyl-6-(3′-(2-PyridylDithio)-Propionamido)-hexanoate). In another embodiment, the linker is a bifunctional chemical linker is that is homobifunctional, examples of which include, without limitation, disuccinimidyl suberate, disuccinimidyl glutarate, and disuccinimidyl tartrate. In other embodiments, the linkers utilized are nucleotide sequences (e.g. DNA) from about e.g. 10 to about 100 nucleotides in length. In yet another embodiments, the linkers utilized may be polysaccharide sequences (e.g., polyglucose, glycosaminoglycan, etc.) that may contain 2 to 100 saccharide units. The linkers may be rigid (e.g. polyproline) or flexible (e.g. various alkyl or modified alkyl chains).

When a linker is present, it is selected so that the two entities that are connected thereby are spaced apart from each other by a distance that is sufficient or adequate to achieve at least some quenching of the fluorescent signal from the fluorescent label attached to the reporter ligand. The amount of quenching sufficient to permit a readily detectable difference between the fluorescent signal from bound vs free reporter ligand could be in the range of 1 to 99% and is preferably in the range of 10 to 90% or in the range of 50-90%. Those of skill in the art will recognize that such a difference will be achieved by modulating the positioning and/or distance between the quenching molecules (the nanoparticle and, optionally, the dye molecule attached to the nanoparticle) and will vary depending on the type of nanoparticle and/or dye that is employed and the type of fluorescent molecule that is used, as discussed elsewhere herein. In some aspects, the linkers are from about 0 to about 500 angstroms in length, or from about 5 to about 100 angstroms in length, or from about 5 to about 50 angstroms in length, in order to permit sufficient quenching.

The linker generally comprises termini that are modified with reactive atoms or groups of atoms that permit covalent attachment (coupling) between the linker and a nanoparticle and/or between the linker and a thrombin molecule and/or between the linker and a dye molecule. For example, if the linker is an alkyl or glycol chain, suitable covalent chemical linkages include but are not limited to those which include a thiol, an ether, an ester, an amide, hydrazine, etc. Exemplary paired reactive terminal moieties that can occur between the thrombin molecule and the nanoparticle include but are not limited to: terminal thiol/dithiolane moieties; terminal hydrazine/carboxyl/amine moieties; terminal alcohol/ether moieties; terminal amide/carboxyl/amine moieties; etc.

### Dye Molecules

In some aspects, the nanoparticle component of the constructs comprise one or more fluorescence quenching dye molecules. The dye molecule(s) function(s) to provide additional quenching (suppression) of fluorescent emission from the detectable label that is attached to the thrombin molecule. Exemplary fluorescence quenching dye molecules include but are not limited to dark quenchers such as various black hole quenchers (BHQs) which quench across the entire visible spectrum; dimethylaminoazobenzenesulfonic acid (dansyl), which absorbs in the green spectrum and is often used with fluorescein; IRDYE® QC-1, which quenches dyes from the visible to the near-infrared range (500-900 nm); QXL™ quenchers, which quench the full visible spectrum; IOWA BLACKK® FQ, which absorbs in the green-yellow portion of the spectrum; Iowa black RQ, which absorbs in the orange-red portion of the spectrum; etc. Suitable dye molecules are selected so as to match the emission wavelengths of the detectable label that is associated with the reporter ligand. For example, for fluorescein (λex=494 nm, λem=521 nm) as the reporter ligand fluorophore, the use of BHQ-1 or BHQ-10 (λmax=516 nm and 534 nm respectively) would be useful.

If a dye molecule is present, it may be attached to the nanoparticle either directly, for example, via a covalent bond, or via a linker molecule, e.g. as described above in the “Linker Molecules” section; or the dye molecule may be attached to the thrombin molecule either directly (e.g. via a covalent bond) or via a linker molecule; or a first dye molecule may be attached to the nanoparticle and a second dye molecule may be attached to the thrombin molecule, also either directly (e.g. via a covalent bond) or via a linker molecule. The first and second dye molecules may be the same or different.

### The Constructs

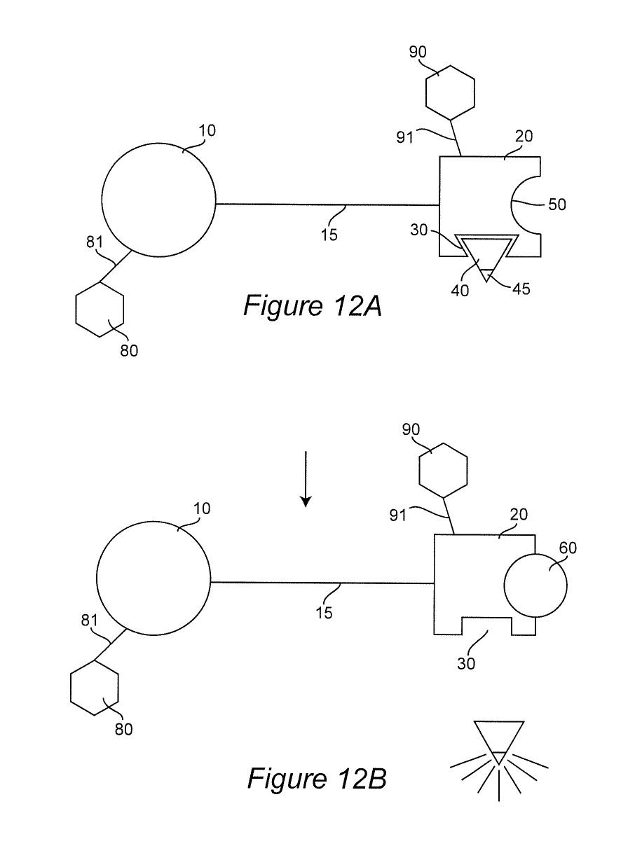

In some aspects, the compositions provided herein are constructs, one example of which (Construct I) is schematically illustrated in FIGS. 12A and B. As can be seen in FIG. 12A, Construct I includes nanoparticle 10 covalently linked to thrombin molecule 20 via (optional) linker 15. Optional dye molecule 80 is connected to nanoparticle 10 via linker 81. Optional dye molecule 90 is connected to thrombin molecule 20 either directly or via linker 81. One or both of dye molecules 80 and 90 may be present in a construct. When one or more dye molecules are present, quenching is via thrombin molecule 20 which comprises binding site 30 and binding site 50. Reporter ligand 40, which comprises fluorescent label 45, is reversibly bound to binding site 30. While bound, no or a decreased fluorescent signal is produced by fluorescent label 45 due to signal quenching by nanoparticle 10 (and/or dye 80 and/or dye 90). When placed in (exposed to, contacted with, etc.) a sample that comprises ligand 60, which is capable of binding specifically or selectively to binding site 50, the binding of ligand 60 to binding site 50 causes a change in binding site 30 so that reporter ligand 40 becomes unbound (FIG. 12B), and a fluorescent signal or stronger fluorescent signal is produced by fluorescent label 45 and can be quantitated. Without being bound by theory, it is believed that in such constructs, the binding of a second ligand to binding site 50 causes a conformational change in the thrombin molecule. The change is transmitted allosterically to binding site 30 and the affinity of binding site 30 for reporter ligand 40 is decreased. Ligand 40 thus tends to leave binding site 30 and be free in solution.

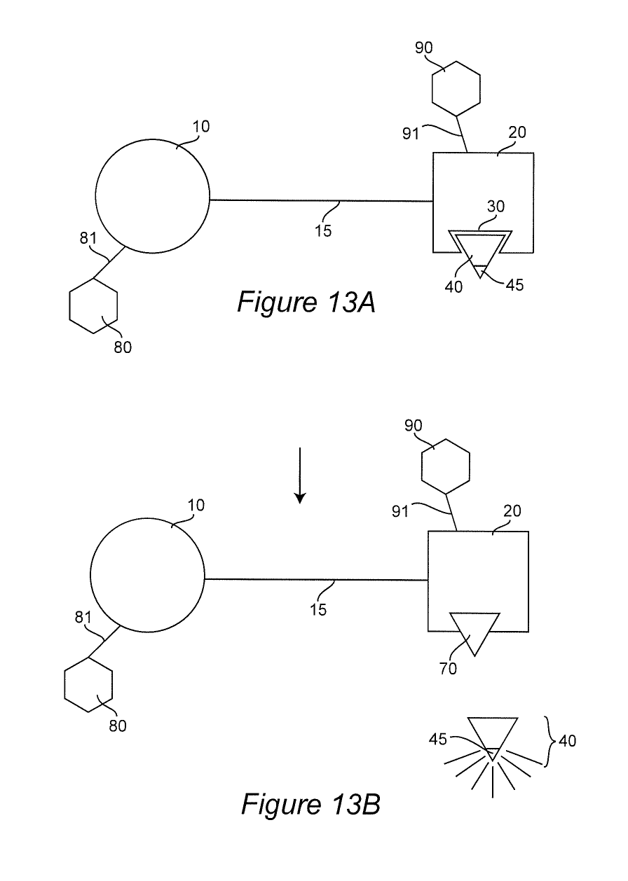

Another aspect of the invention, Construct II, is illustrated in FIGS. 13A and B. In Construct II, thrombin molecule 20 is attached to nanoparticle 10 via (optional) linker 15, and comprises binding site 30 but may or may not comprise additional binding sites (no additional binding sites are shown in exemplary FIG. 13A). Optional dye molecule 80 is connected to nanoparticle 10 via linker 81. Optional dye molecule 90 is connected to thrombin molecule 20 either directly or via linker 81. One or both of dye molecules 80 and 90 may be present in a construct. Reporter ligand 40, which comprises fluorescent label 45, is reversibly bound to binding site 30 and no or a decreased fluorescent signal is produced by fluorescent label 45 due to signal quenching by nanoparticle 10 and/or dye 80 and/or dye 90. When the construct is exposed to ligand 70, ligand 70 replaces or displaces reporter ligand 40 from binding site 30. Reporter ligand 40 is then free in solution and a stronger, detectable fluorescent signal is produced by fluorescent label 45 (FIG. 13B). Without being bound by theory, it is believed that ligand 70 has an affinity for binding site 30 that is significantly higher (e.g. at least about 2-fold higher, and possibly 10, 100 or even 1000-fold higher) than the affinity of reporter ligand 40. Reporter ligand 40 is thus competitively displaced by ligand 70.

Samples and Thrombin-Binding Molecules that are Detected

The methods of the invention can detect a wide variety of thrombin-binding molecules in a wide variety of samples in vivo, ex vivo and in vitro. Exemplary samples include but are not limited to various biological samples that are removed from a subject, including blood, plasma, urine, mucous, cells, tissue samples, etc. If such assays are conducted in vitro or ex vivo, an automated assay may be used which employs, e.g. multi-well (e.g. 96-well) plate, a microchip plate, a strip, or in another type of container which is suitable for use in automated analysis of multiple samples. Alternatively, containers for assessing one sample at a time may be provided, such as a disposable tube for bedside or point-of-care testing. In either scenario, what is provided is a container comprising a plurality of constructs as described herein in e.g. a buffered solution. During use, a defined quantity of a biological sample is placed in the container, allowed to react, and the fluorescent output is measured, e.g. by an automated fluorescence detector, or on the spot, e.g. using a portable fluorescence detection device. The latter aspect of the invention is very useful, for example, in critical care, in operating theatres, and in emergency settings and in follow-up monitoring of critically ill patients since elaborate equipment is not required.

In other aspects, the constructs are used, for example in research, e.g. for screening candidate thrombin-binding molecules, to perform quality control assays of manufactured thrombin-binding molecules, to identify new functions of the potential thrombin-binding molecules.

In all aspects, one or more suitable reference values or controls is/are generally provided as a standard of comparison, e.g. one or more negative samples in which no thrombin-binding molecules are present, and/or one or more positive controls in which a known quantity of a thrombin-binding molecule of interest is present.

Thrombin binding and/or thrombin regulating molecules that may be detected using the constructs and methods disclosed herein include but are not limited to: thrombin inhibitors such as antithrombin (AT), alpha-1-antitrypsin (α-1A), antiplasmin, protease nexin 1, neuroserpin, heparin cofactor II, heparin, hirudin, variegin, desirudin, heparin, heparansulfate, unfractionated heparin (UFH), low molecular weight heparin (LMWH), ultralow molecular weight heparins (ULMWH), heparin pentasaccharide (H5), fondaparinux (FPX), idraparinux (IPX), synthetic heparin analogs; coagulation factors and proteins such as factor VIII, factor V, factor XI, thrombomodulin, glycoprotein Ibalpha, PAR-1 and PAR-4; anticoagulant drugs such as argatroban, bivalirudin, ximelagatran, melagatran, dabigatran; as well as inflammation related proteins such as protein C, factor XII; etc.

### Exemplary Applications of the Technology

As discussed above, the present technology is readily adaptable for bed-side point of care (POC) use in a patient-specific manner. The tests are simple to use (requiring minimal training to interpret), require very small quantities of biological fluid for each assessment (e.g. less than 1 cc) and can be performed rapidly, e.g. in less than 5 minutes. The tests are also readily adapted to “real-time” usage, e.g. during a surgical procedure, to monitor levels of a thrombin-binding molecule on an ongoing basis throughout the procedure. For example, the tests enable monitoring (and hence titrating) AT levels during rapid intravenous administration, providing advancement in cardiac surgery, ICU care, shock treatment, sepsis, eclampsia/pre-eclampsia as well as other usages.

The drug monitoring aspects of this nano-particle monitoring technology permits an increase in the usage of direct thrombin inhibitors and other drug classes such as anti-Xa drugs. Clinical medicine laboratories at hospitals do not presently have available tests of drug levels for these agents. Instead the plasma levels of argatroban, hirudin, bivalirudin and dabigatran (direct thrombin inhibitors) must be performed by specialized laboratories using individualized liquid chromatography. Therefore, when the drugs are used clinically they are used with either surrogate tests (such as activated partial thromboplastin time-APTT, fast thrombin time-TT, thromboelastography-TEG, or RoTEM). These tests have basic inaccuracies and again are not widely utilized. For example the APTT is unreliable as a monitor and the one more often used—the ecarin clotting time is not available in the United States.

The present invention allows wide spread use of drug monitoring of these agents, thereby improving safety and changing (increasing) the indications for usage of these drugs. For example, the use of bivalirudin in heart surgery has been impacted by practitioners' fear of using too little drug, and consequently, there has been a tendency to overdose patients. Bivalirudin has no antidote to reverse its effects. With implementation of the constructs and methods described herein physicians and perfusionists involved in heart surgery can rapidly assess and titrate bivalirudin levels, knowing its half-life (25 minutes), and reduce the risks for post-operative bleeding. Similarly, outpatient use of the oral drug dabigatran can also be titrated and the risks of unexpected bleeds and/or the production and propagation of thrombi lessened or eliminated thereby.

### Detection of the Thrombin Inhibitor Antithrombin (at)

In some aspects, the thrombin-binding molecule, or activity thereof, that is detected and quantitated is antithrombin (AT). There are many reasons for measuring AT activity. For example, antithrombin (AT) activity levels, especially low levels of AT activity, are indicative of or associated with a number of serious diseases and conditions. Low levels of activity may be caused by low levels of AT, or by the production of aberrant or mutant AT molecules with decreased activity. The present tests can be utilized to determine levels of AT activity in samples from individuals at risk of having or developing such diseases/conditions, e.g. in order to diagnose, confirm or further inform a diagnosis of such AT-associated diseases/conditions.

In some instances, AT is administered to patients diagnosed with a condition or disease associated with or caused by low AT activity levels, in order to prevent or reverse symptoms of such diseases. The tests described herein can be used to monitor AT levels prior to, during and after AT administration.

In yet other instances, AT is administered when low levels of AT activity are not necessarily at issue, but when it is desired to promote anticoagulation, e.g. to prevent clotting for medical purposes.

Examples of each of these applications are described below. However, those of skill in the art will recognize that many other instances exist in which it is advantageous to measure AT activity, and the constructs described herein may be utilized in any such application.

Heart Surgery: Worldwide, the number of operative heart surgery cases is 1.5-2.0 million per year. In the United States alone, the number ranges from about 340,000-550,000. During heart surgeries, AT is utilized as an anticoagulant for many patients, possibly in excess of 150,000 cases in the US alone. The technology described herein would fill an unmet need to monitor AT levels in such patients. For example, the test would be used multiple times throughout the procedure, e.g. prior to, during and after surgery. For example, a baseline would be measured prior to AT administration, after AT administration but before surgery, during and/or immediately post-surgery, during recovery, and during follow-up care.

Sepsis: According to the National Institutes of General Medical Sciences data for 2012, 750,000 patients per year in the US were diagnosed with sepsis. During sepsis, plasma levels of antithrombin are very low and are independent predictors of the clinical outcome. A substantial drop in the level of circulating antithrombin has been demonstrated to be a very early phenomenon in sepsis, lending support to the idea that this protease inhibitor is involved in the pathogenesis of the disease. Antithrombin (AT) has thus been used to successfully treat disseminated intravascular coagulation (DIC) occurring during sepsis. However, there is at present no way to conveniently, rapidly and accurately monitor AT levels in sepsis patients. The present disclosure advantageously provides assays which can be used to do so.

Pre-Eclampsia/Eclampsia: Pre-eclampsia, a condition occurring during pregnancy, is characterized by hypertension and increased amounts of protein in the urine. It is thought to affect around 6-8% of all pregnancies. The causes of pre-eclampsia and severe pre-eclampsia are not well understood. If not properly treated, pre-eclampsia can lead to eclampsia which, in turn, can result in seizures and even death. A hallmark of pre-eclampsia is low levels of AT, with pregnancy-induced antithrombin deficiency seen more often in twin and triplet pregnancies. For example, the tests disclosed herein may be used prophylactically or prior to pregnancy, e.g. to identify women at risk of developing pre-eclampsia and to evaluate the degree or level of risk involved. Alternatively, or in addition, the tests may be used to determine AT activity levels after pregnancy is detected, throughout pregnancy, and/or after delivery, as necessary for the benefit of the mother and child. If low levels of AT activity are detected, AT can be administered to prevent and treat pre-eclampsia, e.g. before, during and/or after pregnancy, and the tests disclosed herein can be utilized to monitor AT levels as needed.

Trauma: Major trauma is the leading cause of morbidity and mortality for Americans under the age of 45 years old. 581,000 individuals are hospitalized each year due to trauma, and 180,000 persons per year die of major trauma. If one takes into account minor trauma, the numbers of afflicted individuals exceeds 8 million per year. Treatment of trauma (e.g. wounds) and the prevention of blood clot formation in patients who have experienced trauma is complicated by differing levels of AT activity in the patients, since AT activity impacts the type and/or amount of treatment that is provided. For example, a person with a known AT deficiency is at higher risk of experiencing a blood clot associated with the trauma, or with treatment of the trauma. The rapid tests described herein enable medical professionals to rapidly and accurately determine AT levels and then adjust treatment protocols accordingly. This ability could be especially useful responding to acute trauma situations, such as those involving battlefield wounds and accidents, since the analysis can be carried out on the spot.

Neonates: For healthy full-term neonates, serum AT levels are typically >50% lower than adult reference values. Newborns do not have the thrombotic tendency noted in adults with similarly reduced values because of simultaneous reductions in their procoagulant levels and perhaps due to a protective role of alpha 2-macroglobulin as a thrombin inhibitor in the neonate and in childhood. Premature infants have even lower serum levels of AT. AT levels in the newborn rise to approximately 60% of that of adult levels 1 month after birth. However, various genetic mutations can influence this level, and the superimposition of serious illnesses can further reduce antithrombin due to increased consumption or decreased production. For example, acute respiratory distress syndrome is a known cause of antithrombin deficiency and itself is a major cause of both morbidity and mortality in the newborn. Extracorporeal membrane oxygenation used in the treatment of respiratory failure can be associated with reduced antithrombin levels and increased thrombotic events. Other causes of acquired reductions of antithrombin in neonates include sepsis, asphyxia, liver disease, and maternal preeclampsia or eclampsia, among others. The constructs described herein are advantageously minimally invasive and can be used to monitor AT activity in newborns.

Liver disease: Synthesis of antithrombin and other physiologically important inhibitors of hemostasis, synthesis of procoagulants, and clearance of activated coagulation factors are all regulated by the liver. Thus, the liver plays a central role in hemostasis and liver disease is known to affect AT activity levels, with the severity of liver disease correlating with reductions in AT antigen levels. These reductions are not only due to impaired synthesis, but also to an element of increased consumption, particularly when additional risk factors, such as sepsis, surgery, and hypotension, are present in patients with chronic liver disease. Patients with acute, massive hepatocellular injury and elevations of liver enzyme levels can often have a significantly larger component of a consumptive process than patients with slowly progressive end-stage liver disease. Because of the decreased synthesis of inhibitors as well as the decreased ability to clear activated coagulation factors, patients undergoing orthotopic liver transplantation predictably develop reduced AT levels. The present tests can be utilized to detect decreases in AT activity, e.g. as indicators of disease, and, if AT is administered to counter the low AT activity level, the tests may be used to monitor this AT therapy for a long as necessary.

Kidney disease: Patients with nephrotic syndrome lose antithrombin in the urine, resulting in reduced plasma levels, and they are at higher risk for thrombotic events. Conversely, patients with inherited antithrombin deficiency may develop renal failure due to renal vein thrombosis or due to glomerular deposition of fibrinogen. The degree of compromise in renal function may be such that these patients need renal replacement therapy.

Furthermore, as renal dysfunction progresses, these patients lose increasing amounts antithrombin in the urine and, thus, become even more prone to develop thrombotic episodes. Monitoring the progress of kidney disease both before and after treatment, whether or not AT is administered as a treatment, is an important aspect of establishing treatment protocols, and may be advantageously done in a rapid and cost-effective manner by using the constructs described herein.

Bone marrow transplantation: Veno-occlusive hepatic disease is seen in patients who undergo bone marrow transplantation, particularly in unrelated-donor transplantations, and it is associated with the development of microthrombi in the terminal hepatic venules. This results in rapid, marked deterioration of liver function, causing a coagulopathy characterized by the reduction in the level of antithrombin and, consequently, significant morbidity and mortality. AT may or may not be administered to treat such disease manifestations, but in either case, the ability to measure the level of AT activity in biological samples from the patient using the constructs described herein is highly advantageous.

Drug-induced reduction in antithrombin levels: Many drugs and therapeutic agents are known to decrease AT activity. For example, heparin is the most widely prescribed intravenous drug and is utilized in cardiac catheterization as well as during many invasive radiologic procedures and for the treatment of deep vein thrombosis (DVT). Heparin administration causes an approximately 30% reduction in AT levels, presumably due to rapid clearance in vivo of heparin-antithrombin complexes. As another example, a large body of literature shows that estrogens/oral contraceptives reduce antithrombin levels, potentially resulting in hypercoagulability. In addition, AT deficiency has also been described as associated with asparaginase therapy, occurring by suppression of AT production in the liver as part of the mechanism of action of this chemotherapeutic agent. The constructs described herein can be used to monitor baseline AT activity levels, and/or AT activity levels at any point in treatment or monitoring of patients with these or any conditions, whether or not AT is administered as therapy.

Hereditary antithrombin deficiency: Hereditary antithrombin deficiency is a disorder of blood clotting. People with this condition are at higher than average risk for developing abnormal blood clots, particularly a type of clot that occurs in the deep veins of the legs (DVT, see above). Affected individuals also have an increased risk of developing a pulmonary embolism (PE), which is a clot that travels through the bloodstream and lodges in the lungs. In hereditary antithrombin deficiency, abnormal blood clots usually form only in veins, although they may rarely occur in arteries. About half of people with hereditary antithrombin deficiency will develop at least one abnormal blood clot during their lifetime. These clots usually develop after adolescence. Other factors can increase the risk of abnormal blood clots in people with hereditary antithrombin deficiency. These factors include increasing age, surgery, or immobility. The combination of hereditary antithrombin deficiency and other inherited disorders of blood clotting can also influence risk. Women with hereditary antithrombin deficiency are at increased risk of developing an abnormal blood clot during pregnancy or soon after delivery. They also may have an increased risk for pregnancy loss (miscarriage) or stillbirth. Individuals with this disorder may produce insufficient amounts of AT, or may produce a normal amount of AT that is defective in activity. Long-term anticoagulant thromboprophylaxis is not recommended in asymptomatic patients with AT deficiency because of the increased risk of haemorrhage. However, treatment guidelines recommend short-term thromboprophylaxis in high-risk clinical settings, including surgery, trauma, and management of pregnancy, labor, and delivery. The goal of treatment for patients with hereditary AT deficiency is an initial increase in AT activity to > or =120% of normal levels followed by maintenance of AT activity at > or =80% of normal levels. The constructs described herein are extremely useful for closely monitoring levels of AT activity in such individuals.

### EXAMPLES

These examples describe the successful production and use of an exemplary thrombin nanoprobe (TNP) comprising gold nanoparticles that are covalently linked to alpha-thrombin. The TNP is a stable nanoparticle that binds inhibitors of thrombin. Thrombin has several binding sites including the catalytic active site, exosites 1 and 2, and the sodium binding site.^{45,46} Antithrombin, antithrombin-heparin complexes and dabigatran bind in thrombin's active site, while other proteins and small molecules including hirudins and its variants and bivalirudin bind in exosite 1. Several other potential inhibitors could bind in exosite 2 as shown recently by glycosaminoglycan mimetics.^{47,48} The TNP works for any of these inhibitors because of thrombin's unique plasticity.⁴⁶ In the aspect discussed in these examples, the TNP is non-covalently linked to the fluorescent probe fluorescein. The probe's fluorescence is quenched in the TNP due to the presence of the gold. When the TNP is present in a medium (e.g. plasma, blood, etc.) and a thrombin inhibitor is also present in (or added to) the medium, binding of the inhibitor to the TNP releases the probe and its fluorescence increases. The increase is proportional to the level of the thrombin inhibitor present in the medium. The increase in fluorescence can be quantified e.g. using a light source, or a laser light beam, of appropriate frequency and photodetector. The direct relationship between the amount of fluorescence increase and level of thrombin inhibitor can be converted into % inhibitor activity.

### Example 1

### Synthesis of Gold Nanoparticles (Au NPs)

Au NPs were synthesized as reported in the literature.⁴⁹ Briefly, 100 mL of 0.5 mM HAuCl₄.3H₂O (Sigma-Aldrich, St. Louis, Mo.) in deionized water was charged in a three-necked 500 mL round bottom flask. The solution was heated to 100° C. in an oil bath under vigorous stirring for 30 min. Next, 10 mL of 150 mM sodium citrate dihydrate solution (0.44 g in 10 mL milli Q water) was added into the above solution at 100° C. The color of the solution changed to purple in 5 min and to ruby red in 10 min. The ruby red colored solution of Au NPs was taken out of oil bath immediately and allowed to reach room temperature. The concentration of the Au NP in solution (0.86 nM) was calculated using Beer-Lambert's law (ε₅₂₀=1.2×10⁷ M⁻¹cm⁻¹).⁵⁰ These sodium citrate-stabilized Au NPs were stable for two weeks at RT.

### Example 2

### Synthesis of Gold-Hydrazine Nanoparticles (Au—NHNH₂ NPs)

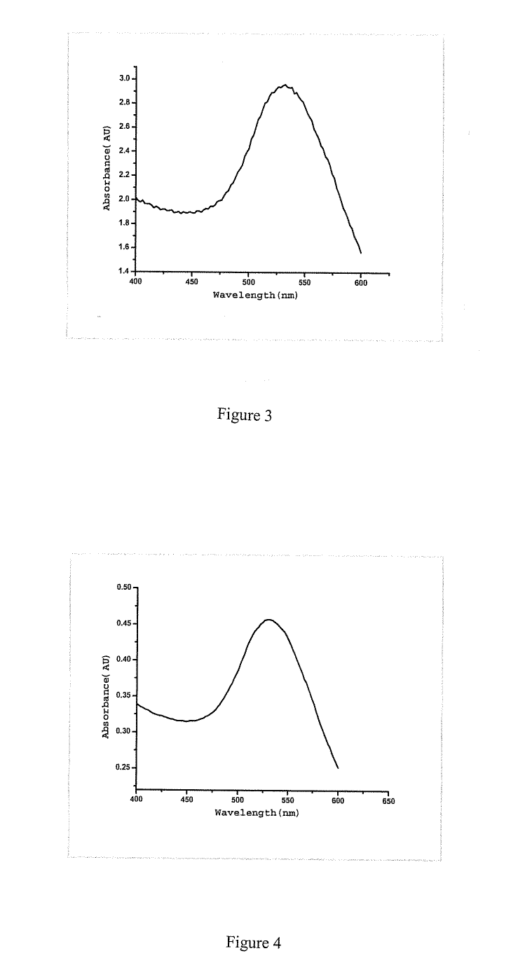

A 10 mL solution of 91.6 nM Au NPs in deionized water was incubated with 500 μL of 1 mM HS-C₁₁EG₃OH (ProChimia Surfaces, Poland) in ethanol and 100 μL of 1 mM HS-C₁₁EG₆OCH₂COOH (ProChimia Surfaces, Poland) in ethanol for three days at 37° C. while constantly shaking. The carboxy-modified Au NPs were purified by centrifugation through MWCO 3000 at 4000 g for 30 min at RT. The centrifugation process was repeated five times using H₂O as eluent. The final volume was made to 1 mL by adding deionized water. The concentration of this solution was found to be 0.33 μM. To modify the carboxy-modified (carboxyl) Au NPs with succinic dihydrazide, the 100 μL of 0.33 μM carboxyl Au NPs were treated with 1 mg 1-Ethyl-3-[3-dimethylaminopropyl]carbodiimide hydrochloride (EDC) (Sigma-Aldrich, St. Louis, Mo.) and 50 μL of 10 mM succinic dihydrazide (Sigma-Aldrich, St. Louis, Mo.) for 2 h at RT in 50 mM PBS buffer, pH 7.4. Excess EDC and succinic dihydrazide were removed by repeated centrifugation through MWCO 3000 at 4000 g for 10 min. The gold hydrazine nanoparticles (Au—NHNH₂ NPs) were washed with deionized water and the volume was made up to 200 μL. The final concentration of the Au—NHNH₂ NPs was found to be 0.24 μM (FIG. 3).

### Example 3

### Thiolation of Bovine α-Thrombin

An aliquot of bovine α-thrombin (200 μg) from a stock solution (8.1 mg/mL, catalog # BCT-1020; Haematologic Technologies, Vermont, USA) was treated with 3-[2-pyridyldithio]propionylhydrazide (PDPH) (17 μg, Thermo Scientific Pierce, Rockford, Ill.) and EDC (6.72 μg, Sigma-Aldrich, St. Louis, Mo.) in a 50 mM phosphate buffer saline (PBS) buffer, pH 7.4 (100 μL) for 2 days at 4° C. while rotating on a rotor. The reaction mixture was then purified and concentrated through centrifugation using a 3000 molecular weight cut-off (MWCO) filter at 10000 g for 10 min. This process was repeated seven times. The final concentration of thiolated bovine α-thrombin was made to 1 μg/μL by adding milli-Q water.

### Example 4

### 5.4 Labelling of α-Thrombin with Black Hole Quencher (BHQ)

α-Thrombin can be linked to a fluorescence quenching dye such as the BHQ-10S by utilizing EDC/NHS (Sulfo-N-hydroxysulfosuccinimide) reaction chemistry. Briefly, a 10 mM stock of BHQ-10S DMF and a 300 μL of 0.1 mg/mL solution of thrombin in PBS buffer pH 7.0 were prepared. 5 μL of the BHQ-10S solution was added to the thrombin and the mixture was vortexed for 30 mins at room temperature, followed by incubation overnight in the refrigerator. The next day, the mixture was loaded to a G-15 matrix sizing column and eluted using PBS buffer pH 7.2, to obtain labeled protein. The protein was concentrated as required.

### Example 5

### Synthesis of Gold-Thrombin (Au-TH) Nanoprobe

### Method A:

Thiolated bovine α-thrombin (100 μg) was incubated with Au—NHNH2 NPs (50 μL, 0.24 μM) and EDC (20 μg) in presence of 50 mM PBS buffer, pH 7.4 (200 μL) for 2 h at room temperature. The bovine α-thrombin stabilized Au NPs were then concentrated through centrifugation using a 10000 MWCO filter at 4000 g for 30 min at 4° C. The final concentration was made to 1 μg/μL by adding 20 mM Tris-HCl buffer, pH 7.4, containing 100 mM NaCl, 2.5 mM CaCl₂ and 0.1% PEG8000 (Buffer A). The absorbance spectrum of Au-thrombin (Au-TH) NPs showed the concentration of gold particles to be 38 nM (FIG. 4).

Method B: Purified carboxy modified AuNPs can be directly linked to thrombin utilizing EDC/NHS coupling reaction. Briefly, 100 μL of a 15 mg/ml solution of EDC and NHS in 10 mM MES buffer pH 5.5 was made fresh before the reaction. 100 μL of concentrated carboxy-modified AuNPs were added to the solution. The mixture was mixed well and incubated for 30 mins at room temperature, following which, 1 mL of MES buffer was added. The solution was centrifuged at 6,500 g for 30 mins to form the activated AuNP pellets. The supernatant was carefully removed and the pellet was washed 1× with MES buffer followed by centrifugation, supernatant removal and another wash with 1 mL of PBS buffer (pH 7.2) containing 0.05% TWEEN®-20 (PBST). The activated AuNPs were centrifuged again, supernatant was removed and 100 μL of 100 g/mL thrombin was added to the AuNPs. The solution was mixed and sonicated in a water bath for 10 seconds followed by incubation for 4 hours at room temperature. After incubation, 1 mL PBST buffer was added, the pellet was washed 2× with PBST via centrifugation at 3,500 g. Tris-HCl was added to quench any unreacted activated carboxyl groups on the AuNP.

### Example 6

### Physical Stability of Au-TH Nanoprobe

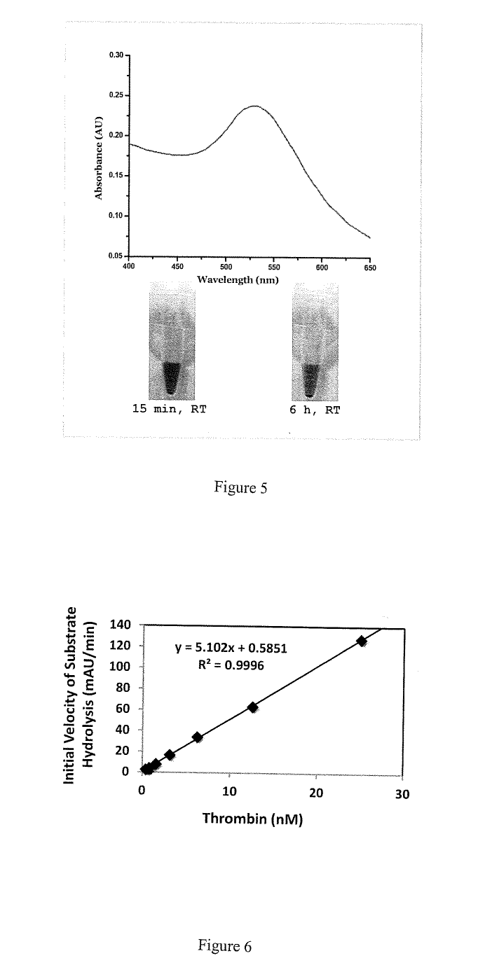

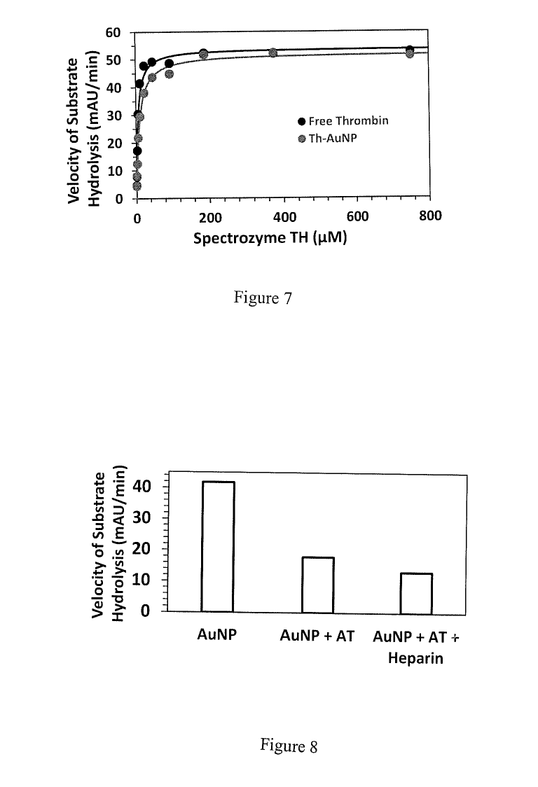

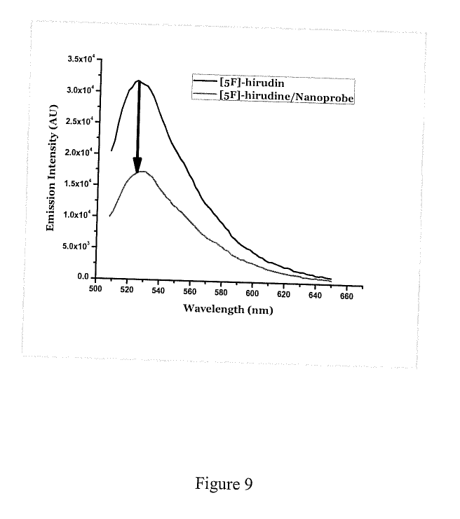

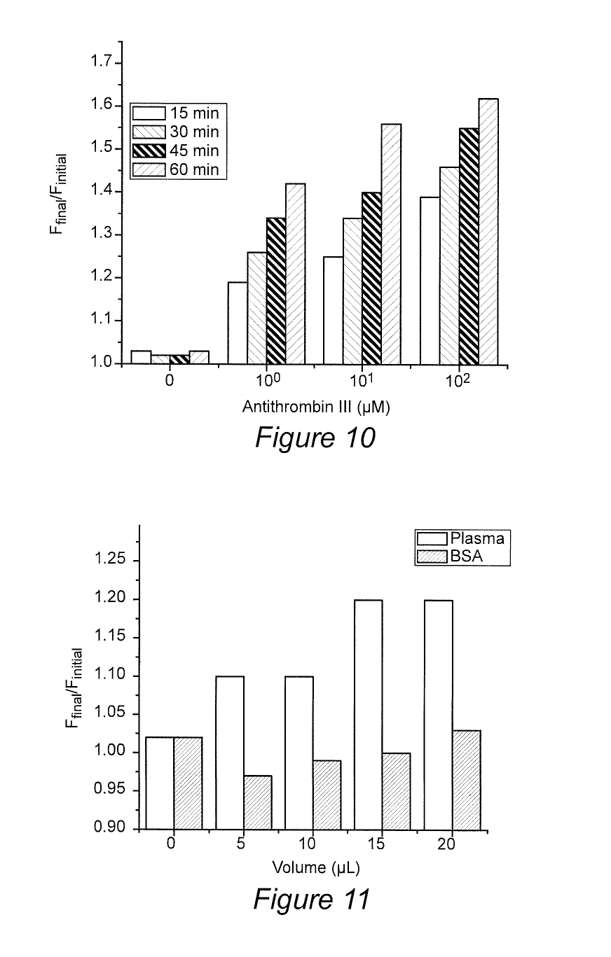

Stability of Au-TH nanoparticles was studied at RT and at −80° C. The NPs were kept in dark at RT for six hours and at −80° C. for 1 month. Absorbance spectra were measured every hour. There was essentially no change in the spectra over the period of six hours confirming the stability of these particles at RT (FIG. 5) Likewise, NPs removed from the low temperature freezer after a month of storage did not exhibit any degradation (not shown).

### Example 7

### Estimation of the Quantity of Thrombin on the Nanoparticle

The amount of thrombin on the surface of the nanoparticle was estimated by the rate of substrate hydrolysis of the Au-TH as compared to standard concentrations of thrombin in buffer. To 40 μL of PBS buffer pH 7.4, 5 μL of 10× diluted Au-Th was added. The solution was incubated at 25° C. for 5 mins, followed by the addition of 5 μL of 2 mM Spectrozyme Th (STh). The velocity of substrate hydrolysis was followed by measuring the slope of the increase in absorbance at 405 nm. The velocity of substrate hydrolysis was then compared to a standard curve (FIG. 4) of known thrombin concentrations and rates to obtain the concentration of thrombin in Au-Th solution. Estimation of number of particles from UV-Visible spectroscopy was then used to determine the number of thrombin molecules per AuNP particle.

### Example 8

### Estimation of the Size of the Nanoparticle Using Dynamic Light Scattering

Dynamic light scattering (DLS) was used to estimate the size of the nanoparticle. Briefly, 400 μL of AuNP solution was placed in a low volume cuvette. The cuvette was inserted into a Malvern Nano S instrument. The solution was allowed to incubate for 2 minutes, followed by a back-scatter reading at 173°. The measurements were taken at 4.65 mm depth of the cuvette. The attenuator and number of runs were set to “auto” to let the instrument determine ideal parameters for the sample. (The solution must have more than 50 kcps scattering intensity for the data to be valid. If it is less than that, a more concentrated solution needs to be sampled.) The intensity scatter was converted to number and volume distributions using the refractive index of gold (n=0.20) and absorption values (k=3.32) respectively.

### Example 9

### Catalytic Activity of Surface Bound Thrombin

Michaelis Menten kinetics were utilized to test the catalytic activity of the surface bound thrombin on the AuNP-TH using a microplate chromogenic substrate hydrolysis assay. Briefly, 165 μL aliquots of PBS buffer pH 7.4 were placed in a 96-well microplate, to which either 5 μL of thrombin at a concentration of 0.04 mg/ml (1 μM) or 5 μL of AuNP-TH was added. The solution was incubated for 5 minutes at 37° C., followed by addition of 30 μL of serial dilutions of Spectrozyme TH substrate such that the final concentrations ranged from 0.7-750 μM. The velocity of substrate hydrolysis was measured by monitoring the slope of the increase in absorbance at 405 nm. A plot of velocity of substrate hydrolysis (v) vs substrate concentration ([S]) (FIG. 5) was fit to the Michaelis Menten equation below to yield the Michaelis Menten constant (K_{M}) and maximum velocity of the reaction (V_{MAX}), which are a direct measure of the catalytic efficiency of the enzyme.

$$v = \frac{{V}_{max} [ S ]}{{K}_{M} + [ S ]}$$

The V_{MAX} and K_{M} remain almost the same as that of free thrombin (FIG. 5) suggesting that the catalytic efficiency of the bound thrombin was not altered significantly by the synthesis protocol of the AuNP-TH.

### Example 10

### Reactivity of Surface Bound Thrombin to Antithrombin (AT)

To test if the surface bound thrombin on the AuNP is reactive towards antithrombin, chromogenic substrate hydrolysis assay like the one mentioned above was performed in the presence of antithrombin and heparin. In a 96-well microplate, 80 μL of PBS buffer was mixed with 5 μL of either AuNP or water. Following this, 5 μL of AT (100 μM) and 5 μL of heparin (3.6 mM) was added and the mixtures were incubated at 25° C. for 20 minutes. Controls were kept with and without AT and heparin, respectively. After incubation, 5 μL of 5 mM Spectrozyme TH was added and the rate of substrate hydrolysis was monitored by observing the absorbance at 405 nm. The velocity of the substrate hydrolysis as obtained by the slope was compared to that of the controls in order to assess the reactivity of the thrombin towards AT (FIG. 6). An approximate 70% reactivity towards AT in the presence of heparin is observed for the AuNP-TH sample.

### Example 11

### Quenching of [5F]-Hirudin (54-65) SO₃⁻ Fluorescence by Au-TH Nanoprobe

To determine the quenching constant of Au-TH nanoprobe, the probe was diluted logarithmically from 10⁻¹ to 10⁻⁷ nM with 20 mM Tris-HCl buffer, pH 7.4, containing 100 mM NaCl, 2.5 mM CaCl₂ and 0.1% PEG8000. This probe (50 μL) was then incubated with 50 μL of 30 nM [5F]-hirudin (54-65) SO₃⁻ (HirP) in a 96-well plate for 0.5 h at RT. HirP is a sulfonated fragment of the 65-residue hirudin peptide. The fragment includes C-terminal amino acids 54 to 65 (GDFEEIPEEYLQ, SEQ ID NO: 4) and bears the fluorescent label fluorescein. Neat HirP (50 μL, 30 nM) was used as positive control (F₀). The 96-well plate was read using a fluorescence plate reader (SYNERGY™ H1 hybrid reader, BioTek instruments) at 525 nm (λₑₓ=488 nm). An approximately 86% quenching of fluorescence from HirP was observed (FIG. 6) suggesting good interaction between HirP and Au-TH nanoprobe. Stern-Volmer plot was prepared by plotting the ratio of the emission intensity (F₀) of neat HirP (50 μL, 30 nM) and the emission intensity (F) of Au-TH nanoprobe containing HirP at equivalent concentration against Au-TH nanoprobe concentration. The quenching constant was determined from the slope of the straight line (not shown).

### Example 12

### Probing Antithrombin (AT) Levels in Buffer

Au-TH-NP (6 nM, 50 μL) and HirP (50 μL, 30 nM) were mixed with 20 mM Tris-HCl buffer, pH 7.4, containing 100 mM NaCl, 2.5 mM CaCl₂ and 0.1% PEG8000 and used as the standard nano-metal surface energy transfer (NSET) pair. Antithrombin (AT) was serially diluted starting from 100 μM to 1 pM by adding 20 mM Tris-HCl buffer, pH 7.4. Serially diluted AT solutions were then incubated with standard NSET pair in a 96-well plate for 0-1 h at RT with gentle rocking. A solution of 100 μL of Au-TH-HirP nanoprobe (NSET pair) mixed with 50 μL of buffer was used a control (F₀). After 1 h of incubation, the 96-well plate was read at 525 nm (λₑₓ=488 nm) (F). A plot of F/F₀ as Y-axis against AT concentration as X-axis is shown as FIG. 5. The results show an approximately 40%, 25 and 20% increase in fluorescence at 525 nm following addition of AT at 1, 10 and 100 μM levels, respectively, at 1 hr incubation time point. The fluorescence increases were smaller at 15, 30 and 45 min timepoints (FIG. 5). Noticeable increases were also found for AT concentrations less than 1 μM (not shown).

### Example 13

### Probing Antithrombin Levels in Plasma

A 96-well plate was used for this experiment. The baseline fluorescence of the NSET pair (Au-TH-NS (6 nM, 50 μL) and HirP (50 μL, 30 nM) in 50 μL of 20 mM Tris-HCl buffer, pH 7.4, containing 100 mM NaCl, 2.5 mM CaCl₂ and 0.1% PEG8000) was measured by recording the fluorescence emission at 525 nm (λₑₓ=488 nm). The NSET pair was then incubated with different volumes of plasma (5 μL, 10 μL, 15 μL and 20 μL). To assess specificity of AT reaction, the NSET pair was also incubated with 600 μM BSA (5 μL, 10 ΞL, 15 μL and 20 μL). The fluorescence intensity was recorded within 1-3 min after incubation began (FIG. 6). The results show an approximately 10%, 10%, 20% and 20% increase in fluorescence at 525 nm following addition of human plasma at 5, 10, 15 and 20 levels, respectively. In contrast, addition of BSA resulted in a significant decrease in emission intensity suggesting its inability to release HirP from the Au-TH nanoprobe.

This example shows that gold nanoparticles functionalized with thrombin that is reversibly labeled with a detectable label can be used to detect thrombin inhibitors in biological samples.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

It should be further noted that the phrase “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e. g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e. g., 46.7-91.3) should also be understood to be intended as possible subrange endpoints unless specifically excluded.

Further, it should be noted that terms of approximation (e. g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

In addition, the adjectives “a” and “the” should not be strictly interpreted as singular but, where appropriate, should be interpreted to refer to plural quantities as well.

We claim:

1. A nanoprobe comprising:
a metal nanoparticle;
a thrombin molecule attached to said metal nanoparticle, said thrombin molecule comprising at least one ligand binding site; and
a reporter ligand non-covalently bound to said at least one ligand binding site, said reporter ligand comprising a fluorescent label;
wherein an absorbance spectrum of said metal nanoparticle overlaps an emission spectrum of said fluorescent label;
and wherein a distance between said metal nanoparticle and said fluorescent label allows quenching of a fluorescence signal from said fluorescent label by said metal nanoparticle.

2. The nanoprobe of claim 1, wherein said thrombin molecule is covalently attached to said metal nanoparticle via a first linker molecule.

3. The nanoprobe of claim 1, further comprising
a dye molecule attached to said metal nanoparticle, or
a dye molecule attached to said thrombin molecule,
or
a dye molecule attached to said metal nanoparticle and a dye molecule attached to said thrombin molecule,
wherein said dye molecule is attached to said metal nanoparticle, or to said thrombin molecule, or to said metal nanoparticle and to said thrombin molecule directly or via a linker molecule,
and wherein an absorbance spectrum of said dye molecule overlaps an emission spectrum of said fluorescent label, and wherein a distance between said dye molecule and said fluorescent label allows quenching of said fluorescence signal from said fluorescent label by said dye molecule.

4. The nanoprobe of claim 1, wherein said reporter ligand comprises hirudin or a thrombin-binding portion of hirudin.

5. The nanoprobe of claim 1, wherein said fluorescent label is selected from the group consisting of fluorescein, cyanine, tetramethylrhodamine, and boron-dipyrromethene (BODIPY®).

6. The nanoprobe of claim 1, wherein said metal nanoparticle is selected from the group consisting of gold, iron, copper, silver, platinum, tungsten and alloys and derivatives thereof.

7. The nanoprobe of claim 1, wherein said thrombin molecule comprises at least a portion of a thrombin protein sequence from a species selected from the group consisting of human, bovine, ovine, porcine, non-human primate and rodent.

8. The nanoprobe of claim 1, wherein said thrombin molecule comprises at least a portion of a thrombin protein sequence selected from the group consisting of an alpha thrombin, a beta thrombin and a gamma thrombin.

9. The nanoprobe of claim 1, wherein said at least one binding site is selected from the group consisting of a thrombin catalytic active site, thrombin exosite 1, and thrombin exosite 2.

10. The nanoprobe of claim 1, wherein said metal nanoparticle is gold, said thrombin molecule is bovine alpha thrombin, and said fluorescent label is fluorescein.

11. A method of detecting a thrombin inhibitor in a biological sample, comprising the steps of
i) contacting said biological sample with a nanoprobe, said nanoprobe comprising:
a metal nanoparticle;
a thrombin molecule attached to said metal nanoparticle, said thrombin molecule comprising at least one ligand binding site; and
a reporter ligand non-covalently bound to said at least one ligand binding site,
said reporter ligand comprising a fluorescent label;
wherein an absorbance spectrum of said metal nanoparticle overlaps an emission spectrum of said fluorescent label; and wherein a distance between said metal nanoparticle and said fluorescent label allows quenching of a fluorescence signal from said fluorescent label by said metal nanoparticle;
and
ii) detecting the presence or absence of a fluorescent signal from said fluorescent label, wherein in the presence of a fluorescent signal indicates that a thrombin inhibitor is present in the biological sample, and the absence of a fluorescent signal indicates that a thrombin inhibitor is not present in the biological sample.

12. The method of claim 11 wherein said thrombin inhibitor detected in said detecting step is selected from the group consisting of antithrombin (AT), alpha-1-antitrypsin (α-1A), antiplasmin, heparin cofactor II, heparin, hirudin, desirudin, argatroban, bivalirudin, ximelagatran, melagatran, dabigatran, heparin, heparansulfate, unfractionated heparin (UFH), low molecular weight heparin (LMWH), ultralow molecular weight heparins (ULMWH), heparin pentasaccharide (H5), fondaparinux (FPX), idraparinux (IPX) and a synthetic heparin analog.

13. The method of claim 11 wherein said detecting step further comprises a step of quantifying said thrombin inhibitor.

14. The method of claim 13 wherein said step of quantifying includes the steps of measuring said fluorescent signal and comparing measured fluorescence to one or more reference values.

15. The method of claim 11 wherein said metal nanoparticle is selected from the group consisting of gold, iron, copper, silver, platinum, tungsten and alloys and derivatives thereof.

16. The method of claim 11 wherein said fluorescent peptide is selected from the group consisting of fluorescein, cyanine, tetramethylrhodamine, and boron-dipyrromethene (BODIPY®).

17. The method of claim 11 wherein said thrombin molecule is from a species selected from the group consisting of human, bovine, ovine, porcine, non-human primate and rodent.

18. The method of claim 11 wherein said thrombin molecule comprises at least a portion of a thrombin protein sequence selected from the group consisting of an alpha thrombin, a beta thrombin and a gamma thrombin.

19. The method of claim 11 wherein said at least one ligand binding site is selected from the group consisting of a thrombin catalytic active site, thrombin exosite 1, and thrombin exosite 2.

20. The method of claim 11 wherein said metal nanoparticle is gold, said thrombin molecule is bovine alpha thrombin, and said fluorescent peptide is fluoroscein.

21. The method of claim 11, wherein said method is performed in vivo.

22. The method of claim 11, wherein said method is performed ex vivo.

23. The method of claim 11, wherein said method is performed in vitro.

24. The method of claim 11, wherein said nanoprobe further comprises
a dye molecule attached to said metal nanoparticle, or
a dye molecule attached to said thrombin molecule,
or
a dye molecule attached to said metal nanoparticle and a dye molecule attached to said metal nanoparticle,
wherein said dye molecule is attached to said metal nanoparticle, or to said thrombin molecule, or to said metal nanoparticle and to said thrombin molecule directly or via a linker molecule,
wherein an absorbance spectrum of said dye molecule overlaps an emission spectrum of said fluorescent label, and wherein a distance between said dye molecule and said fluorescent label allows quenching of said fluorescence signal from said fluorescent label by said dye molecule.


SEQUENCE LISTING (4 sequences)

SEQ ID NO: 1 - protein, 259 aa, Homo sapiens
IVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 2 - protein, 105 aa, Homo sapiens
GQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 3 - protein, 65 aa, Hirudo medicinalis
  MISC_FEATURE: tyrosine at position 63 is sulfated
VVYTDCTESGQNLCLCEGSNVCGQGNKCILGSDGEKNQCVTGEGTPKPQSHNDGDFEEIPEEYLQ

SEQ ID NO: 4 - protein, 15 aa, Hirudo medicinalis
GDFEEIPEEYLQSEQ